(19) United States
(12) Patent Application Publication
(10) Pub. No.: US 2011/0178410 A1
(43) Pub. Date: Jul. 21, 2011

(54) Method of Quantitative Analysis Utilizing Multiphoton Microscopy

(75) Inventors: Chen-Yuan Dong, Taipei City (TW); Wen Lo, Taipei City (TW); Sung-Jan Lin, Taipei City (TW); Shiou-Hwa Jee, Taipei City (TW); Ruei-Jr Wu, Taipei City (TW)

(73) Assignee: NATIONAL TAIWAN UNIVERSITY, Taipei City (TW)

(21) Appl. No.: 12/690,705

(22) Filed: Jan. 20, 2010

(51) Int. Cl. A61B 6/00

(52) U.S. Cl. 600/476

(57) ABSTRACT

The present invention utilizes multiphoton microscopy for a quantitative analysis of a bio tissue or a skin. Multiphoton microscopy is characterized in low invasion, low photo damage and high penetration. Hence, scanning through multiphoton microscopy does not hurt the bio tissue. It is thus fit for scanning live bio tissues to know its aging status. In addition, the quantitative analysis provides a precise index for diagnosing a damage severity of the tissue, like cancer.

### FIELD OF THE INVENTION

The present invention relates to an analysis utilizing multiphoton microscopy; more particularly, relates to a clinical quantitative analysis for bio tissue and aging skin.

### DESCRIPTION OF THE RELATED ARTS

Clinically, distribution of a bio tissue is an important factor on diagnosing the bio tissue for its wellness. Yet, the distribution of the bio tissue is traditionally diagnosed by experience without knowing an acute pathological level. For obtaining cellular physiological data in the bio tissue while penetrating through a possible good thickness, section with staining is a method providing an image with a high resolution. However, this method only provides a photo at a 2-dimensional level at one time, while an image at a 3-dimensional level may thus be ruined. At the same time, the sectioned tissue has to be fixed after some chemical processes, which is not possible for a live tissue. Additionally, this method can be used only when an obvious pathological sign appears, where a best early treatment may be missed. Hence, the prior arts do not fulfill all users' requests on actual use.

### SUMMARY OF THE INVENTION

The main purpose of the present invention is to provide a precise pathologic index of a bio tissue showing a damage severity of the bio tissue in a short time for an early treatment.

The second purpose of the present invention is to utilize multiphoton microscopy with low invasion, low photo damage and high penetration for not hurting the bio tissue and for improving practicability of a clinical diagnosis.

The third purpose of the present invention is to greatly reduce damage over the bio tissue with only central symmetric tissues, like collagen in cornea, shown in the image and a clear image obtained by not showing unnecessary tissues.

To achieve the above purposes, the present invention is a method of a quantitative analysis utilizing multiphoton microscopy, comprising steps of: (a) scanning a bio tissue with a pulsed laser obtained through a multiphoton microscope; (b) receiving an auto-florescence image of the bio tissue; and (c) processing a quantitative analysis of the auto-florescence image to obtain an index. Accordingly, a novel method of a quantitative analysis utilizing multiphoton microscopy is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the scanning;

FIG. 3 is the view showing the photoaging indexes; and

FIG. 4 is the view showing the cancer indexes.

### DESCRIPTION OF THE PREFERRED EMBODIMENT

The following description of the preferred embodiment is provided to understand the features and the structures of the present invention.

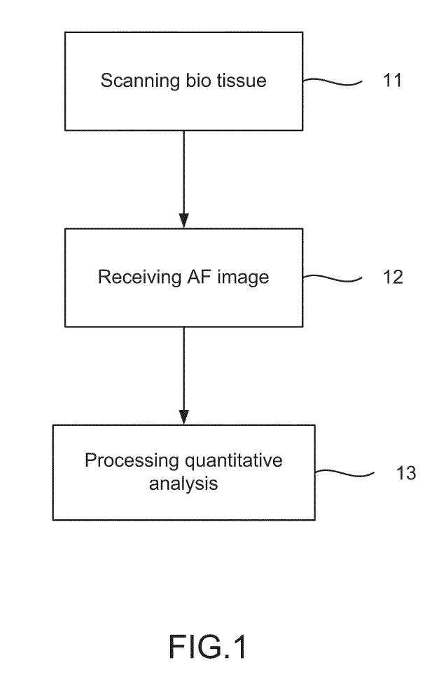

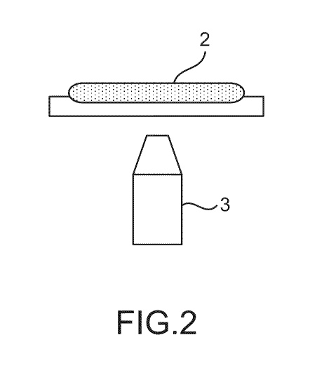

Please refer to FIG. 1 and FIG. 2, which are a flow view showing a preferred embodiment according to the present invention and a view showing a scanning. As shown in the figures, the present invention is a method of a quantitative analysis utilizing multiphoton microscopy, comprising the following steps:

(a) Scanning bio tissue 11: As shown in FIG. 2, multiphoton microscopy is utilized. A multiphoton microscope 3 is used to scan a bio tissue 2 with a pulsed laser of 1000 nanometers generated.

(b) Receiving auto-florescence (AF) image 12: An AF image of the bio tissue thus obtained is received.

(c) Processing quantitative analysis 13: The AF image of the bio tissue is processed through a quantitative analysis to obtain an index.

On using the present invention, multiphoton microscopy is utilized for scanning the bio tissue. Then, the AF image thus obtained is analyzed. Finally, the index is figured out through the following steps:
(i) A fluorescence intensity of a second harmonic generation (SHG) image of the bio tissue is subtracted from a fluorescence intensity of the AF image.
(ii) The fluorescence intensity of the SHG image is added to the fluorescence intensity of the AF image.
(iii) The result of the subtraction is divided by the result of the addition to figured out the index.

The index figured out, denoted as SAAID, is also called a photoaging index with a formula of

$$SAAID = \frac{A F - S H G}{A F + S H G} .$$

An absolute base for a clinical diagnosis is thus obtained with the SAAID.

In addition, the AF image can be replaced by a third harmonic generation (THG) image of the bio tissue; and the index, denoted as STID, is thus figured out with a formula of

$$STID = \frac{T H G - S H G}{T H G + S H G} .$$

Because the THG process does not directly excite the bio tissue, it has less harm to the bio tissue than the AF process. Hence, practicability of a clinical diagnosis is improved.

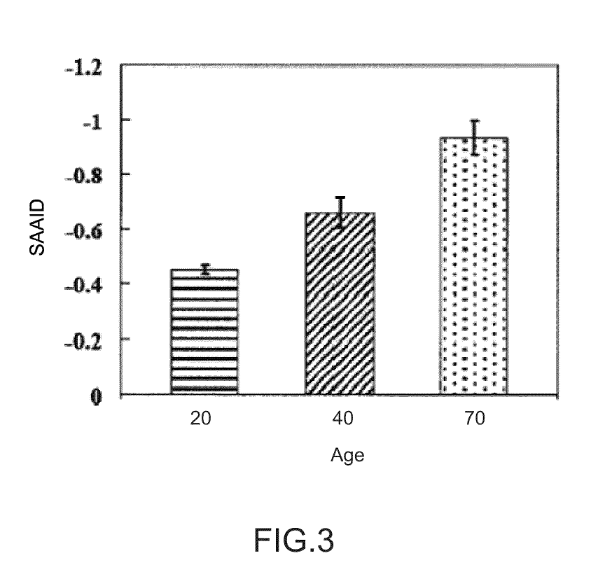

Please refer to FIG. 3, which is a view showing photoaging indexes. As shown in the figure, the present invention is used to diagnose photoaging status for medicine applied on skin. When skin is sunned or aging naturally, elastic fiber increases and collagen decreases. According to the present invention, a fluorescence image is obtained through a non-invasive scan of SHG utilizing multiphoton microscopy. Then a quantitative analysis is processed to obtained a precise photoaging index (SAAID). As shown in the figure, index for aging skin is greater than index for young skin.

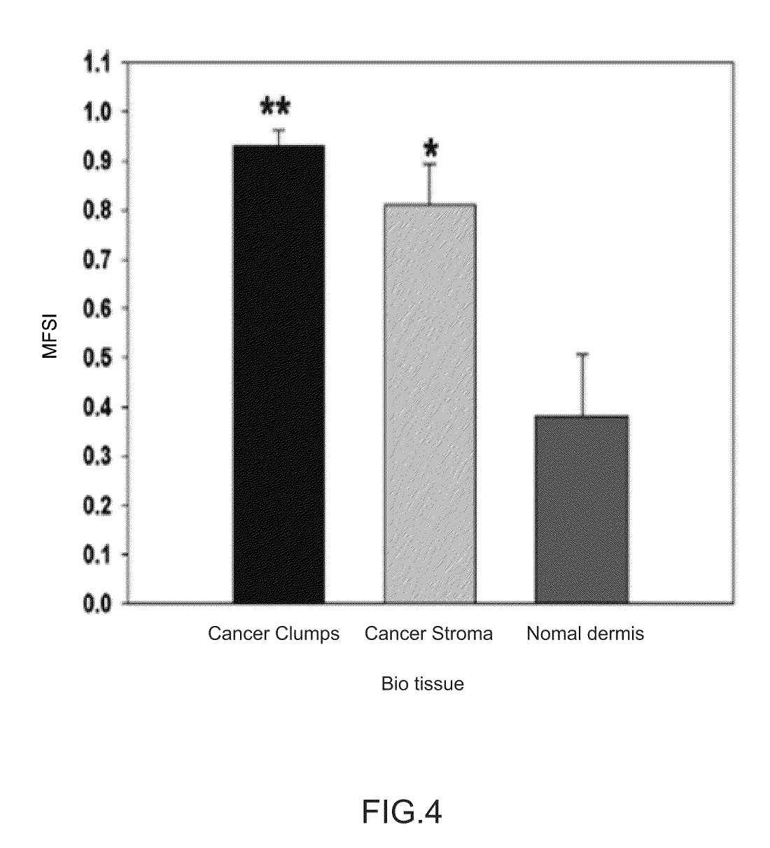

Please refer to FIG. 4, which is a view showing cancer indexes. As shown in the figure, the present invention is used to diagnose cancer. Under an SHG scanning through multiphoton microscopy, a fluorescence image of a normal tissue shows more collagen, while cancer tissue has less or even none collagen and thus lots of holes shows in the fluorescence image of the cancer tissue. After a quantitative analysis according to the present invention, precise cancer index (MFSI) is obtained; and, as shown in the figure, index of cancer tissue is obviously higher than index of normal tissue.

The present invention utilizes multiphoton microscopy with low invasion, low photo damage and high penetration for not hurting a bio tissue and for improving practicability of a clinical diagnosis. On processing a THG scanning on the bio tissue, damage over the bio tissue is greatly reduced. Moreover, only central symmetric tissues, like collagen in cornea, are shown in the image; so that a clear image is obtained by not showing unnecessary tissues. In addition, through the quantitative analysis according to the present invention, a value of a damage severity (i.e. polarity) of a bio tissue is defined; and, an objective and precise index for diagnosing the bio tissue is thus provided.

Accordingly, the present invention obtains a clear fluorescence image without hurting a live bio tissue. Through a quantitative analysis, a value of a damage severity of the bio tissue is further defined while totally preventing human factor of misdiagnosis. Hence, a reliable base for clinically diagnosing the bio tissue is provided to obtain the damage severity of the bio tissue in a short time for an early treatment.

To sum up, the present invention is a method of a quantitative analysis utilizing multiphoton microscopy, where a clear fluorescence image is obtained without hurting a bio tissue; a value of a damage severity of the bio tissue is defined through a quantitative analysis while totally preventing human factor of misdiagnosis; and, a reliable base for clinically diagnosing a bio tissue is thus provided to obtain the damage severity of the bio tissue in a short time for an early treatment.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of a quantitative analysis utilizing multiphoton microscopy, comprising steps of:
(a) scanning a bio tissue with a pulsed laser of 1000 nanometers obtained through a multiphoton microscope;
(b) obtaining an auto-florescence image of said bio tissue; and
(c) processing a quantitative analysis of said auto-florescence image of said bio tissue to obtain an index.

2. The method according to claim 1, wherein said index is obtained by:
(i) subtracting a fluorescence intensity of a second harmonic generation (SHG) image of said bio tissue from a fluorescence intensity of said auto-florescence (AF) image of said bio tissue;
(ii) adding said fluorescence intensity of said SHG image of said bio tissue to said fluorescence intensity of said auto-florescence image of said bio tissue; and
(iii) dividing the result of said subtraction by the result of said addition; and
wherein said index, denoted as SAAID, is thus obtained with a formula of
$$SAAID = \frac{A F - S H G}{A F + S H G} .$$

3. The method according to claim 1,
wherein said AF image of said bio tissue is replaced by a third harmonic generation (THG) image of said bio tissue and said index, denoted as STID, is thus obtained with a formula of
$$STID = \frac{T H G - S H G}{T H G + S H G} .$$
